(19) United States
(12) Patent Application Publication
(10) Pub. No.: US 2014/0017339 A1
(43) Pub. Date: Jan. 16, 2014

(54) MATERIALS AND METHODS FOR TREATING RADIATION POISONING

(71) Applicants: Louis M. Pelus, Carmel, IN (US); Jonathan Hoggatt, West Lafayette, IN (US)

(72) Inventors: Louis M. Pelus, Carmel, IN (US); Jonathan Hoggatt, West Lafayette, IN (US)

(73) Assignee: Indiana University Research and Technology Corp., Indianapolis, IN (US)

(21) Appl. No.: 13/883,460

(22) PCT Filed: Nov. 7, 2011
(86) PCT No.: PCT/US2011/059619

§ 371 Date: Sep. 30, 2013

Related U.S. Application Data
(60) Provisional application No. 61/410,814, filed on Nov. 5, 2010.

(51) Int. Cl. A61K 33/24; A61K 31/42; A61K 31/5415; A61K 31/191

(52) U.S. Cl. CPC A61K 33/24; A61K 31/191; A61K 31/42; A61K 31/5415
USPC 424/646; 514/573; 514/378; 514/226.5

(57) ABSTRACT

Disclosed herein are materials and methods for treating patients that have been exposed to pathological levels on ionizing radiation. These materials and methods include administering to a patient in thereof at least one dose of a compound such as prostaglandin (PGE₂) as soon as possible after the patient has been exposed to the radiation. Additional embodiments include treating a patient exposed to radiation with at least one dose of a compound that modulates PGE₂ activity such as non-steroidal anti-inflammatory compound. Some methods include a first step of administering PGE₂ or another compound that binds as soon as possible after exposure to radiation followed by a delayed dosing with at least one compound such as meloxicam that interferes with PGE₂ activity.

### PRIORITY CLAIM

This patent application claims the benefit of U.S. Provisional Patent Application No. 61/410,814, filed on Nov. 5, 2010, which is incorporated herein by reference in its entirety.

### STATEMENT OF GOVERNMENTAL RIGHTS

This invention was made with government support under grant number HL 069669 and under grant number HL096305 awarded by the National Institutes of Health. The United States Government has certain rights in the invention.

### FIELD OF THE INVENTION

This invention relates generally to materials and methods for treating humans or animals that have been exposed to pathological levels of ionizing radiation.

### BACKGROUND

Given increases in the proliferation of nuclear weapons, the use of nuclear power, and the real risk of radical terrorism, there is an increasing need for countermeasures for use in the event of a radiological mass casualty event (Poston, Sr., 2005; Pellmar and Rockwell, 2005; Moulder, 2004). Chief among these measures are to develop effective therapies for treating radiation poisoning. Radiation poisoning, also sometimes referred to as radiation syndrome or radiation sickness, is caused by exposure to high levels of ionizing radiation. The condition is often lethal and there are few effective treatments for this condition save supporting the patient and protecting the patient from exposure to infectious agents which can readily decimate the immuno-compromised body of patients suffering from radiation poisoning.

Exposure to high levels of radiation disproportionally affects rapidly dividing cells and systems comprised of rapidly dividing cells. Symptoms of radiation poisoning include nausea, diarrhea, headache, hair loss, skin sloughing, anemia and a severely compromised immune system. One specific effect of radiation poisoning is its effect on Hematopoietic stem cells (HSC) and hematopoietic progenitor cells (HPC). Under normal conditions these cell types are constantly proliferating in order to supply the upwards of a trillion cells a day (Ogawa, 1993) needed to maintain homeostasis in for example an adult human being.

The highly proliferative nature of hematopoietic stem (HSC) and progenitor (HPC) cells, particularly during stress induced hematopoiesis, makes them highly sensitive to radiation, and in extreme circumstances results in the Hematopoietic Syndrome of the Acute Radiation Syndrome (HS-ARS). HS-ARS is characterized by life-threatening neutropenia, thrombocytopenia and lymphocytopenia, and possible death due to infection and/or bleeding. While HSC and HPC are susceptible to radiation exposure, surviving populations of these cells can recover hematopoiesis if given critical time to repair DNA damage, self-renew, expand and differentiate. This constant state of proliferation make HSC and HPC highly radiosensitive (Hall, 2000b; Chinsoo and Glatstein, 1998), meaning that most, if not all, successful radiation poisoning countermeasures will need to either address or prevent radiation induced damage to the hematopoietic system. Some aspects of the invention disclosed herein address some of these needs.

### SUMMARY

Some aspects of the invention provide methods for treating radiation poisoning comprising the steps of administering a therapeutically effective dose of prostaglandin E2 (PGE₂), wherein the first dose is administered as soon as possible after the patient has been exposed to radiation. A further step includes administering a dose of at least one compound that interferes with the function of PGE₂. These compounds include anti-inflammatory NSAIDs compounds such as meloxicam (4-hydroxy-2-methyl-N-(5-methyl-2-thiazolyl)-2H-1,2-benzothiazine-3-carboxamide-1,1-dioxide), which is sometimes sold under the trade name Mobic.

Some aspects of the invention include methods of treating the patient suffering from radiation poisoning, comprising the steps of: administering a therapeutically effective dose of PGE₂ or of a compound that has the same or a similar physiological effect as PGE₂ to a patient soon after said patient has been exposed to a pathological dose of ionizing radiation; and dosing the patient with a therapeutically effective amount of at least one compound that reduces the activity of PGE₂, wherein there is a delay between the administering step and said dosing step.

Some embodiments include methods wherein the effective dose of PGE₂ or a compound that has a similar effect on HSC or HPC cells as PGE₂ is administered within 24 hours of the patient's exposure to the dose of ionizing radiation. In some embodiment the effective dose of PGE₂ or a compound that has an effect similar to PGE₂ is administered within 12 hours of the patient's exposure to the dose of ionizing radiation.

In some embodiments, the patient suffering from radiation poisoning is given at least one therapeutically effective dose of at least one compound that reduces the activity of PGE₂ about 24 hours after the patient has been exposed to the pathological dose of ionizing radiation. In some embodiments, the patient is given a dose of a compound that reduces the production of or efficacy of PGE₂ at least about 36 hours after the patient has been exposed to the pathological dose of ionizing radiation and after the patient has been given at least one dose of a compound with PGE₂ or PGE₂ like activity. In some embodiments, the compound that reduces the production of or efficacy of PGE₂ is given to the patient at least about 48 hours after the patient has been exposed to the pathological dose of ionizing radiation.

Some embodiments of the invention include administering a therapeutically effective amount of 16,16 dimethyl prostaglandin E₂ (dmPGE₂), or a pharmaceutically acceptable salt thereof. In some embodiments, the therapeutically effective dose of dmPGE₂ is in the range of about 10.0 to about 0.01 mg per kg⁻¹, in some embodiments the individual doses of dmPGE₂, or compounds with comparable activity, ranges from about 10.0 to about 0.01 mg per kg⁻¹, in still other embodiments the dose ranges from about 5.0 to about 0.08 mg per kg⁻¹, and in still other embodiments the doses range from about 2.0 to about 0.1 mg per kg⁻¹. Still other embodiments include treating a patient in need thereof with a therapeutically effective dose of at least one compound selected from the group consisting of: PGE₁, PGE₃, dmPGE₁, and dmPGE₃.

Some embodiments include administering a therapeutically effective dose of at least one compound which alters the activity of PGE₂ and wherein the dose of the compound is given after the patient is treated with a therapeutically effective dose of PGE₂ or a compound that acts on HSC and/or HPC in a manner similar to how PGE₂ acts on HSC and/or HPC. In some embodiments, the compound that alters the activity of PGE₂ acts on at least one enzyme selected from the group consisting of: cyclooxygenase-1 and cyclooxygenase-2. In some embodiments the compound that alters PGE₂ activity acts primarily on cyclooxygenase-2.

In some embodiments, the non-steroidal anti-inflammatory compound is selected from the group consisting of: aspirin, celecoxib, rofecoxib, etoricoxib, valdecoxib, ibuprofen, naproxen, diclofenac, etodolac, ketorolac, indomethacin, meloxicam and licofelone and pharmaceutically acceptable salts thereof. In some embodiments, the NSAID is indomethacin or a pharmaceutically acceptable salt thereof. And, in still other embodiments, the NSAID is meloxicam or a pharmaceutically acceptable salt thereof. In some embodiments of the invention, the therapeutically effective doses of said NSAID are in the range of about 100.0 to about 0.01 mg per kg⁻¹ per day, in some embodiments the individual doses of NSAID is in the range of about 75.0 to about 0.05 mg per kg⁻¹ per day, and in still other embodiments the doses of NSAIDs range from about 74 to about 0.08 mg per kg⁻¹ per day, and in some embodiments the kg⁻¹ doses of NSAID are in the range of about 50 to about 0.1 mg per kg⁻¹ per day.

In some embodiments, the compound that alters the activity of PGE₂ and is administered to a patient suffering from acute radiation poisoning is an antagonist of at least one PGE₂ receptor. These compounds are administered to the patient after treatment with at least one molecule that has PGE₂ activity or PGE₂ like activity towards HSC and HPC. In some embodiments, the antagonist of at least one PGE₂ receptor used to practice the invention is selected from the groups consisting of: N-[[4′-[[3-butyl-1,5-dihydro-5-oxo-1-[2-(trifluoromethyl)phenyl]-4H-1,2,4-triazol-4-yl]methyl][1,1′-biphenyl]-2-yl]sulfonyl]-3-methyl-2-thiophenecarboxamide and 4-(4,9-diethoxy-1,3-dihydro-1-oxo-2H-benz[f]isoindol-2-yl)-N-(phenylsulfonyl)-benzeneacetamide.

Some aspect of the invention include methods for treating radiation poisoning, comprising the steps of: administering a therapeutically effective course of treatment, the course including doses of at least one compound that increases Hif-1α activity. In some embodiments the course of treatment with the compound that increases Hif-1α activity is started within about 24 hours of the patient's exposure to ionizing radiation and in still other embodiments it may be started with about 12 hours or even with about 6 hours after exposure to pathological levels of radiation. In some embodiments the course of treatment may comprise a single dose of the compound while in other embodiments that compound may be administered to the patient in a series of doses.

In some embodiments the compound that increases Hif-1α activity is cobalt chloride (CoCl₂). Some embodiments include methods of treating radiation poisoning comprising the step of treating a patent in need thereof with CoCl₂. In some embodiments the patient may be treated with a single dose of the compound while in other embodiments therapeutic amounts of CoCl₂ may be administered to the patient incrementally. In some embodiments of the invention the therapeutically effective dose of CoCl₂ is about between about 120.0 to about 5.0 mg per kg⁻¹ per day. In still other embodiments the therapeutically effective kg⁻¹ dose of CoCl₂ is about between about 100.0 to about 10.0 mg per kg⁻¹ per day or between about 60.0 to about 20.0 mg per kg⁻¹ per day. In still other embodiment, the methods for treating radiation poisoning include administering a therapeutically effective dose of CoCl₂ which is administered to the patient within 24 hours of the patient's exposure to ionizing radiation. In some embodiments, the therapeutically effective dose of CoCl₂ is administered within 6 hours of exposure to ionizing radiation.

### BRIEF DESCRIPTION OF THE FIGURES

FIG. 1. Bar graph illustrating that non-steroidal anti-inflammatory drugs increase progenitor cell expansion.

FIG. 2A. Graph of percent survival measured followed for 30 days after LD₅₀ irradiation.

FIG. 2B. Bar graph showing total CFC (CFU-GM, BFU-E and CFU-GEMM) measured 30 days after LD₅₀ irradiation.

FIG. 3A. Graph of percent survival as a function of time followed for 30 days after LD₉₅ irradiation.

FIG. 3B. Bar graph summarizing colonies per femur determined for different cell types measured 30 days after LD₉₅ irradiation.

FIG. 4A. White Blood Cell (WBC) counts (K mL₋₁) as a function of time determined 30 days after LD₉₅ irradiation and measured for mice that were not treated (control) or treated with meloxicam, meloxicam 2 or dmPGE₂.

FIG. 4B. Polymorphonuclear cell (PMN) counts (K μL⁻¹) as a function of time determined 30 days after LD₉₅ irradiation and measured for mice that were not treated (control) or treated with meloxicam, meloxicam 2 or dmPGE₂.

FIG. 4C. Platelet (PTL) counts (K μL⁻¹) as a function of time, determined 30 days after LD₉₅ irradiation and measured for mice that were not treated (control) or treated with meloxicam, meloxicam 2 or dmPGE₂.

FIG. 5A. A bar graph of total CFC per femur measured in mice 30 days after irradiation. The mice were treated as follows: no treatment (control); treatment with CoCl₂, dmPGE₂, or meloxicam.

FIG. 5B. Graph percent survival of irradiated animals measured as function of days for mice that were not treated after being irradiated (control) and mice that were treated after irradiation with CoCl₂.

### DESCRIPTION

For the purposes of promoting an understanding of the principles of the novel technology, reference will now be made to the preferred embodiments thereof and specific language will be used to describe the same. It will nevertheless be understood that no limitation of the scope of the novel technology is thereby intended, such alterations, modifications, and further applications of the principles of the novel technology being contemplated as would normally occur to one skilled in the art to which the novel technology relates are within the scope of this disclosure and the claims.

As used herein, unless explicitly stated otherwise or clearly implied otherwise, the term ‘about’ refers to a range of values plus or minus 10 percent, e.g., about 1.0 encompasses values from 0.9 to 1.1

As used herein, unless explicitly stated otherwise or clearly implied otherwise, the terms ‘therapeutically effective dose,’ ‘therapeutically effective amounts,’ and the like, refer to a portion of a compound that has a net positive effect on the health and well being of a human or other animal. Therapeutic effects may include an improvement in longevity, quality of life, and the like. These effects also may include a reduced susceptibility to developing disease or deteriorating health or well being. The effects may be immediate realized after a single dose and/or treatment or they may be cumulatively realized after a series of doses and/or treatments.

Determining the optimal dosing level and/or course of treatment for a given human or animal patient is dependent on a number of well known factors including the age, size, species, and health of the human or animal patient being treated. Given, data for a group of mammalian patients, it is well within the skills of the clinician of ordinary skill in a given medical or veterinary specialty to determine an advantageous dosing level or course, barring any unexpected results, without having to engage in undue experimentation.

Dosing units as used herein are generally given in units of mass of the active ingredient per kilogram of the patient's or donor's body mass, e.g., mg of NSAID per kg (mg kg⁻¹) of the patient's body mass.

Pharmaceutically acceptable salts include salts of active ingredients used to practice the invention that are generally considered to be safe and effective for use in mammals and that may also possess a desired therapeutic activity. Pharmaceutically acceptable salts include salts of acidic or basic groups present in compounds of the invention. Exemplary pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain compounds of the invention may form pharmaceutically acceptable salts with various amino acids. Suitable base salts include, but are not limited to, aluminum, calcium, lithium, magnesium, potassium, sodium, zinc, and diethanolamine salts. For additional information on some pharmaceutically acceptable salts that can be used to practice the invention, please see reviews such as Berge, et al., 66 J. PHARM. SCI. 1-19 (1977), Haynes, et al, J. Pharma. Sci., Vol. 94, No. 10, October 2005, pgs. 2111-2120 and the like.

Some aspects of the invention include using compounds that can reduce the effect of PGE₂ on HSC and HPC cells. These compounds include, but are not limited to, wherein the compound that reduces PGE₂ activity is a non-steroidal anti-inflammatory compound. This class includes compounds that act on at least one enzyme selected from the group consisting of: cyclooxygenase-1 and cyclooxygenase-2. In some embodiments, the steroidal anti-inflammatory compound acts primarily on cyclooxygenase-2. In some embodiments, the non-steroidal anti-inflammatory compound is selected from the group consisting of: aspirin, celecoxib, rofecoxib, etoricoxib, valdecoxib, ibuprofen, naproxen, diclofenac, etodolac, ketorolac, indomethacin, meloxicam and licofelone. In some embodiment, the non-steroidal anti-inflammatory compound is indomethacin. While in others, it may be or may include meloxicam.

In some embodiments, the compound that alters the activity of PGE₂ is an antagonist of at least one PGE₂ receptor. In some embodiments, these antagonists of at least one PGE₂ receptor may be selected from the groups consisting of: N-[[4′-[[3-butyl-1,5-dihydro-5-oxo-1-[2-(trifluoromethyl)phenyl]-4H-1,2,4-triazol-4-yl]methyl][1,1′-biphenyl]-2-yl]sulfonyl]-3-methyl-2-thiophenecarboxamide and 4-(4,9-diethoxy-1,3-dihydro-1-oxo-2H-benz[f]isoindol-2-yl)-N-(phenylsulfonyl)-benzeneacetamide.

For additional information see, for example, International Publication Number WO 2010/054271 A1 based on International Application No. PCT/US2009/063654, having an International Filing Date of Nov. 6, 2009 entitled “Materials and Methods to Enhance Hematopoietic Stem Cells Engraftment Procedures” to Pelus, et al., which itself claims the benefit of U.S. provisional patent application No. 61/112,018 filed on Nov. 6, 2008 and also see, for example, International Publication No. WO 2011/060381, based on International Application No. PCT/US2010/056744, having an International Filing Date of Nov. 15, 2010, entitled “Methods to Enhance Delivery and Engraftment of Stem Cells Including the Identification of Specific Prostagandin E₂ Receptors”, to Pelus, et al., which itself claims the benefit of U.S. provisional patent application No. 61/261,352 filed on Nov. 15, 2009 and U.S. provisional patent application No. 61/261,349 filed on Nov. 15, 2009, each of these International Patent Applications and U.S. provisional patent applications are incorporated herein by reference in its entirety as if each were incorporated individually in its entirety.

Regulation of hematopoiesis at this radiation sensitive stage is controlled through accessory cell produced cytokines and growth factors and interactions with the microenvironmental niche stromal cells themselves (Broxmeyer and Smith, 2009; Shaheen and Broxmeyer, 2009). It is known that radiation damage to the hematopoietic system occurs both at the levels of HSC and HPC and through changes to cells within the marrow microenvironment that provide signals for their self-renewal, proliferation, survival, differentiation, and migration (Broxmeyer et al., 1976; Coleman et al., 2003; Dainiak et al., 2003; MacVittie et al., 2005; Till and McCulloch, 1964), and that substantive damage to bone marrow causes the hematopoietic syndrome of the acute radiation syndrome (HS-ARS).

HS-ARS is characterized by life-threatening lymphocytopenia, neutropenia, and thrombocytopenia, and possible death due to infection and/or bleeding. Doses <2 Gy do not cause significant bone marrow damage (Anno et al., 1989); however, at doses of 2-8 Gy, the acute radiation syndrome develops proportional to radiation dose, resulting in development of cytopenias and marrow failure in ensuing weeks post exposure (Hall, 2000b; Chinsoo and Glatstein, 1998; Wald, 1982), with the resultant sequela of infection, bleeding and deficient wound healing, in the absence of treatment (Dainiak et al., 2003; Coleman et al., 2003). While bone marrow HSC and HPC are susceptible to radiation exposure, surviving populations of these cells can recover hematopoiesis, given critical time to repair DNA damage, self-renew, expand and differentiate.

The unpredictability of a mass casualty radiation event requires development and utilization of post exposure mitigators of radiation injury with appropriate ease of administration, stability for purposes of stockpiling, ability for rapid distribution and a window of efficacy. In addition, faced with the complexities of a mass casualty event and difficulty of individual dosimetry and triage, interventions that can mitigate or reduce the severity of exposure but that are benign to those individuals with limited or no exposure are required.

As disclosed herein, treatment with dmPGE₂ shortly following irradiation or delayed administration of meloxicam post-irradiation results in significantly enhanced hematopoietic recovery and survival. By way of explanation and not limitation and without being bound by any single theory or hypothesis, one explanation consistent with the results disclosed herein is as follows: Many proffer radio-protecting agents, including PGE₂, are thought to act by increasing hypoxia (Allalunis-Turner et al., 1989; Purdie et al., 1983; Glover et al., 1984).

The potential for HIF-1α modulation to act as a radio-mitigation was investigated. The effects on HSC by treatment PGE₂ were measured. Administering PGE₂ decreased apoptosis through up-regulation of Survivin, and increased homing/engraftment in bone marrow transplants. International Publication Number WO 2010/054271 A1 based on International Application No. PCT/US2009/063654. Results, not shown, demonstrate that reducing PGE₂ signaling by inhibiting PGE₂ biosynthesis with NSAID administration results in a rapid expansion of HPC in bone marrow. Similar strategies based on modulating PGE₂ signaling post-irradiation could both protect HSC from radiation induced apoptosis and/or lead to quicker hematopoietic recovery, resulting in increased survival.

### Exemplary Experiments and Results

### Mice

Male and female C57B1/6 mice were purchased from Jackson Laboratories at 10-12 weeks of age. Mice were housed in microisolator cages (5 mice per cage) with sterilized direct contact bedding (Alpha Dri). Animal holding rooms were maintained at 21±3° C. with 30 to 80% relative humidity, with at least 10 air changes per hour of 100% fresh air, and a 12-hour light/dark cycle. Mice were fed ad libitum with commercial rodent chow (Harlan 2018SXC) in cage hoppers and acidified (pH 2.0-3.0) water in sipper tube bottles. The Institutional Animal Care and Use Committee of IUSM approved all protocols.

### Effect of PGE₂ on Proliferation

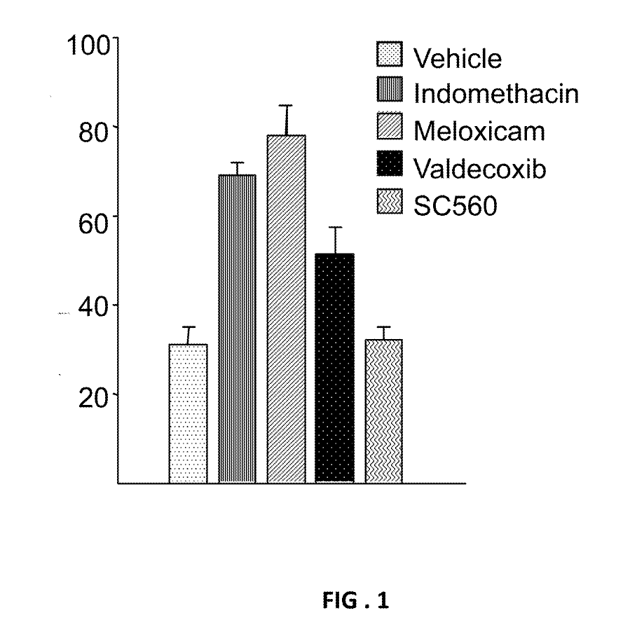

Mice were administered dual (Indomethacin, meloxicam), COX2 selective (Valdecoxib) or COX1 selective (SC560) inhibitors twice daily for 4 days. Referring now to FIG. 1, on day 5, mice were sacrificed, bone marrow from one femur isolated, total nucleated cells counted on a Hemavet 950FS and CFU-GM determined. Data are Mean±SEM for N=5 mice per group, each assayed individually.

### Irradiation

Mice were placed in single chambers of a plexiglass irradiation pie (Braintree), with 15 mice per pie, alternating groups of males and females within the same pie. The mice in each group of mice, irradiated together in the same pie, were divided equally among all treatment groups to ensure that each group received the same irradiation exposure conditions. Mice were irradiated between 9:00 a.m. and 11:00 a.m. from a ¹³⁷Cesium gamma radiation source (GammaCell 40; Nordion International, Kanata, Ontario, Canada) at an exposure rate ˜63 cGy per minute, and received 796 cGys total exposure.

### Effects of PGE₂ and NSAIDs on LD₅₀ Irradiated Mice

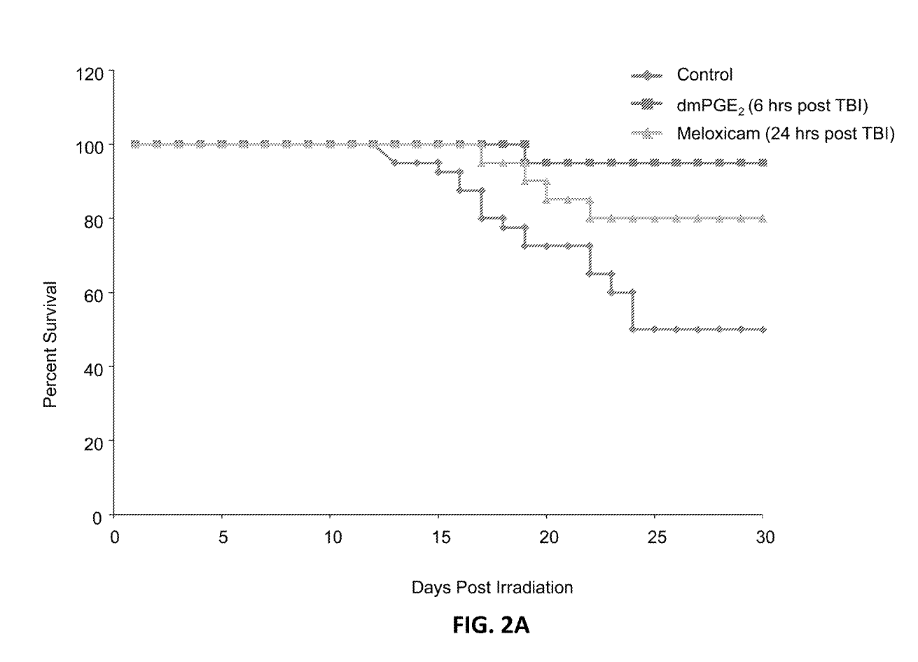

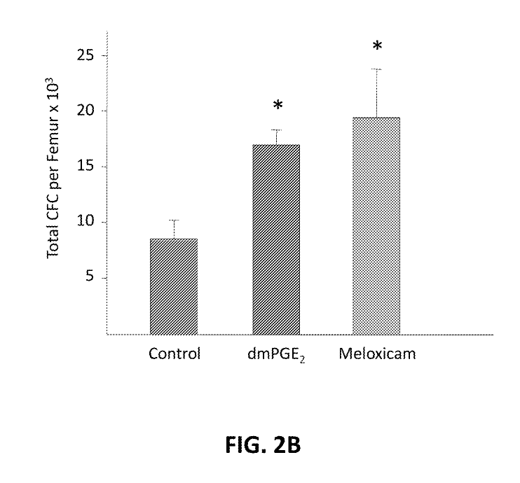

Cohorts of 20-40 mice were irradiated with an LD₅₀ dose from a ¹³⁷Cs source. Mice received a single dose of dmPGE₂ (40 μg/mouse) at 6 hours post-irradiation, or 6 mg/kg meloxicam dosed daily on days 2-5 post-irradiation. Animals were monitored for morbidity and mortality twice/daily for 30 days and euthanized when moribund. Referring now to FIG. 2A, survival curves were analyzed with a log-rank test.

After 30 days, analysis of total CFC(CFU-GM, BFU-E and CFU-GEMM) was performed in methylcellulose culture. Referring now to FIG. 2B, data are Mean±SEM from N=5 mice per group, each assayed individually.

### Effects of PGE₂ and NSAIDs on LD₉₅ Irradiated Mice

Cohorts of 20 mice were irradiated with an LD₉₅ dose from a ¹³⁷Cs source. Mice received a single dose of dmPGE₂ (40 μg/mouse) at 6 hours post-irradiation, or 6 mg/kg meloxicam dosed daily for 4 days starting 6 hours post-irradiation (meloxicam) or 2 days post-irradiation (meloxicam 2). Animals were monitored for morbidity and mortality twice/daily for 30 days and euthanized when moribund.

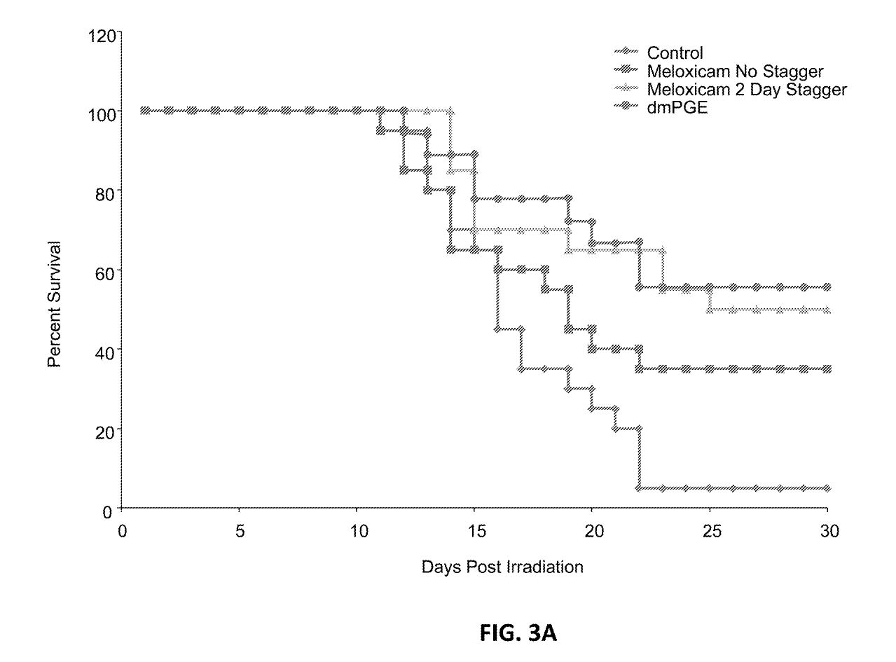

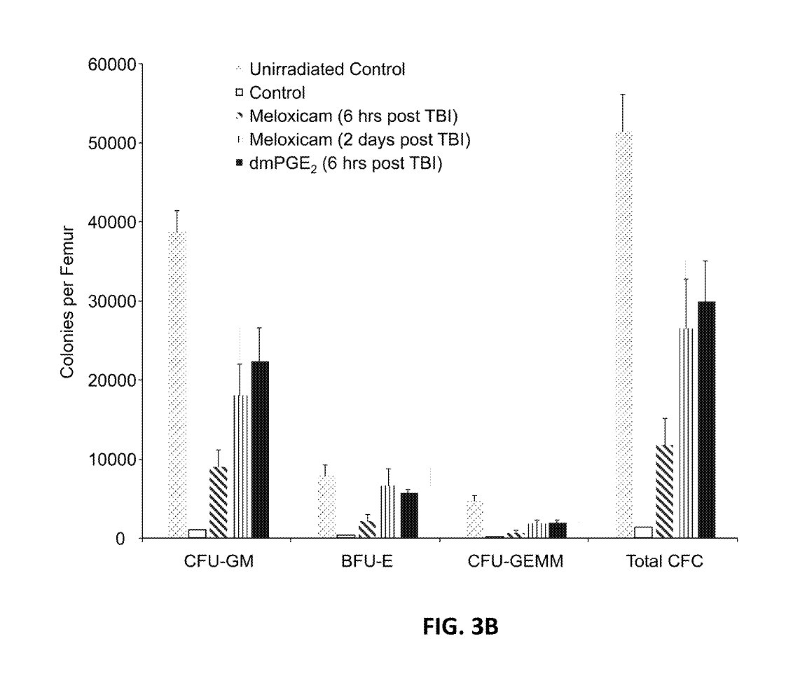

Referring now to FIG. 3A, survival curves were analyzed with a log-rank test (Bottom of Second Column). Mice were followed for 30 days. Survival was measured for mice treated as follows: no treatment (control); treatment with dmPGE₂ administered soon after irradiation; meloxicam administered soon after irradiation; and meloxicam administered 48 hours after irradiation. Referring now to FIG. 3B, this graph summarizes the total colony forming units recovered from the femurs of mice that were irradiated and then treated as follows: un-irradiated (control), irradiated control; treated with meloxicam either 6 hours or 24 hours post-irradiation and treated with dmPGE₂ 6 hours after irradiation.

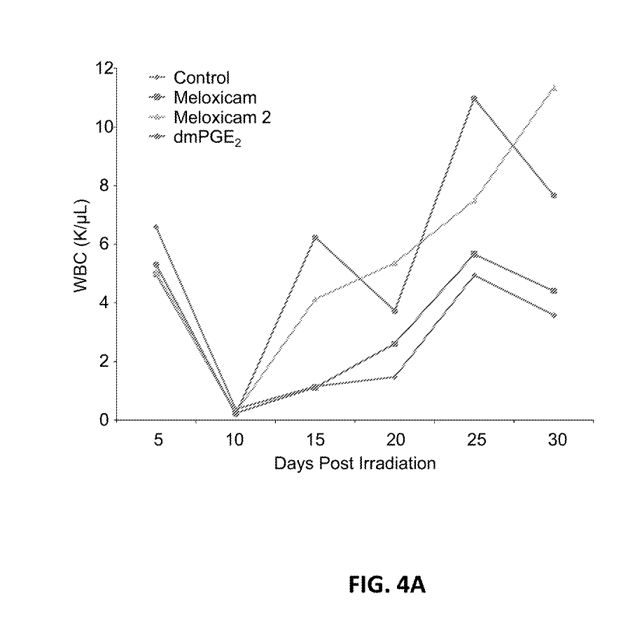

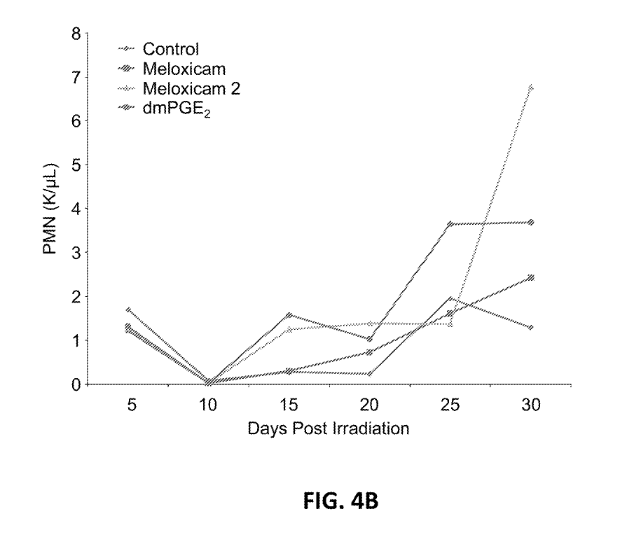

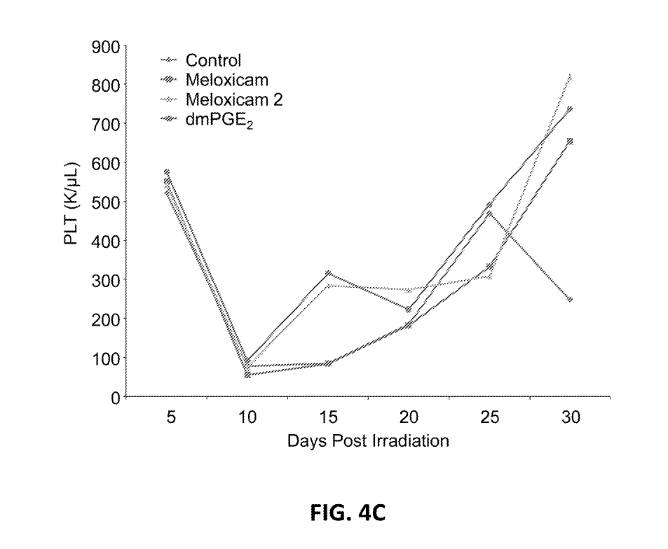

Referring now to FIGS. 4A, 4B and 4C. Every 5 days, 50 μL of blood was acquired from 3 random mice from each treatment cohort and white blood cell (WBC), polymorphonuclear cell (PMN) and platelet (PLT) counts were determined (Left).

After 30 days, analysis of total CFC(CFU-GM, BFU-E and CFU-GEMM) was performed in methylcellulose culture and compared to a non-irradiated control group. Referring now to FIG. 3B, data are Mean±SEM from N=3 mice per group, each assayed individually.

### Treatment Post-Irradiation

Irradiated mice were identified by ear punches and treated with either a single subcutaneous dose of dmPGE₂ (40 μg/mouse) or vehicle control at 6 hours post-irradiation (N=20 mice per group, evenly split male/female); 6 mg/kg meloxicam dosed once daily on days 2 through 5 post-irradiation or vehicle control (N=20 or 40 mice per group, respectively, evenly split male/female); or a single subcutaneous dose of CoCl₂ (60 mg/kg) at 6 hours post-irradiation (N=20 mice per group, evenly split male/female). One mouse per treatment group was housed in each individual cage.

### Morbidity and Mortality Monitoring

Mice were observed for morbidity or mortality once daily during the acclimation period and twice daily starting on the day after irradiation for thirty days. Moribund mice were scored for signs of early euthanasia based on three parameters: the severity of hunched posture, squinted/closed eyes, and decreased activity. Each criterion was scored on a scale of 1 to 3, with 3 being the most severe. Moribund mice with a score of 8 or 9 were euthanized and the date of death was recorded.

### Colony Assays

After 35 days, remaining irradiated mice were sacrificed, bone marrow acquired from femurs, and total CFC including CFU-GM, BFU-E and CFU-GEMM were enumerated in 1% methylcellulose/IMDM containing 30% HI-FBS, 1 U/ml rhEPO, 10 ng/ml rhGM-CSF and 50 ng/ml rmSCF as described (Broxmeyer et al., 2007; Fukuda et al., 2007). All cultures were established in triplicate from individual animals, incubated at 37° C., 5% CO₂, 5% O₂ in air for 7 days and colonies quantified by microscopy.

### PGE₂ Treatment Increases Survival Post-Irradiation

PGE₂ biosynthesis is increased following γ-radiation and can result from up-regulation of cPLA₂ (Chen et al., 1996) or COX2 (Isoherranen et al., 1999). In rats, spinal cord irradiation elevates PGE₂ levels within 3-24 hours that persist for 3 days (Siegal and Pfeffer, 1995). In mice, brain irradiation induces COX-dependent PGE₂ production and elevated levels of PGE₂ synthases (Moore et al., 2005). In breast cancer patients, radiation therapy triggers monocyte PGE₂ production (Cayeux et al., 1993) and in leukemia and lymphoma patients undergoing autologous transplant, plasma PGE₂ levels were 3-12 fold higher than controls between days 0 and 10 post-transplant (Cayeux et al., 1993). High PGE₂ levels occurred when patients were cytopenic, suggesting that PGE₂ was produced by cells less sensitive to cytoreductive therapies. Due to the anti-apoptotic and self-renewal properties of PGE₂ signaling, it is possible that up-regulation of PGE₂ synthesis is an endogenous mechanism for radioprotection although it is unclear from the literature that this is the case. In the absence of the examples reported herein, it was also plausible that the increased expression of PGE₂ after exposure to high levels of ionizing radiation was a consequence of radiation poisoning that occurred without advantage to the afflicted patient or even to the detriment of the patient's health or chances for survival. And although PGE₂ is endogenously produced as a consequence of radiation damage, exogenous administration of PGE₂ or other compounds that mimic its activity in the body, particularly metabolically stable dmPGE₂ analogs, are likely to be more efficacious and maintain higher levels of active PGE₂ for longer periods, thereby magnifying whatever positive affect exogenously produced PGE₂ has on HSC survival and function. Early studies have explored the use of dmPGE₂ administered prior to radiation exposure (Hanson and Ainsworth, 1985; Hanson, 1987; Walden, Jr. et al., 1987; Walden, Jr. and Farzaneh, 1995); however, in the case of a mass casualty event, prophylactic administration is not feasible, and little research has explored the use of dmPGE₂ post-irradiation as a “radiomitigator” rather than a “radioprotector”.

As disclosed herein, PGE₂ was able to mediate damage done by irradiation. Referring now to FIGS. 2A and 2B, these figures illustrate the radio-protective effects of various drug regimes used to treat mice after LD₅₀ irradiation. Percent survival was determined using the following treatment regime: no treatment (control); with dmPGE₂ administered 6 hours after irradiation; and treatment with meloxicam administered 24 hours after irradiation. Referring now to FIG. 2B, a bar graph showing Total CFC per femur measured after LD₅₀ irradiation cells were collected from mice that were: not treated (control); treated with dmPGE₂; or treated with meloxicam.

Irradiated mice with therapeutic exogenously provided amounts of PGE₂ mitigated the damage to hematopoietic cells observed post-irradiation with dmPGE₂ treatment, using a murine HS-ARS model developed by Dr. Orschell at IUSM. Cohorts of 20-40 mice were irradiated with an LD₅₀ dose from a ¹³⁷Cs source. Mice received a single dose of dmPGE₂ (40 μg/mouse) at 6 hours post-irradiation, or 6 mg/kg meloxicam dosed daily on days 2-5 post-irradiation. Animals were monitored for morbidity and mortality twice/daily for 30 days and euthanized when moribund. Survival curves were analyzed with a log-rank test.

Referring now to FIG. 2B, after 30 days, analysis of total CFC(CFU-GM, BFU-E and CFU-GEMM) was performed in methylcellulose culture. Data are Mean±SEM from N=5 mice per group, each assayed individually.

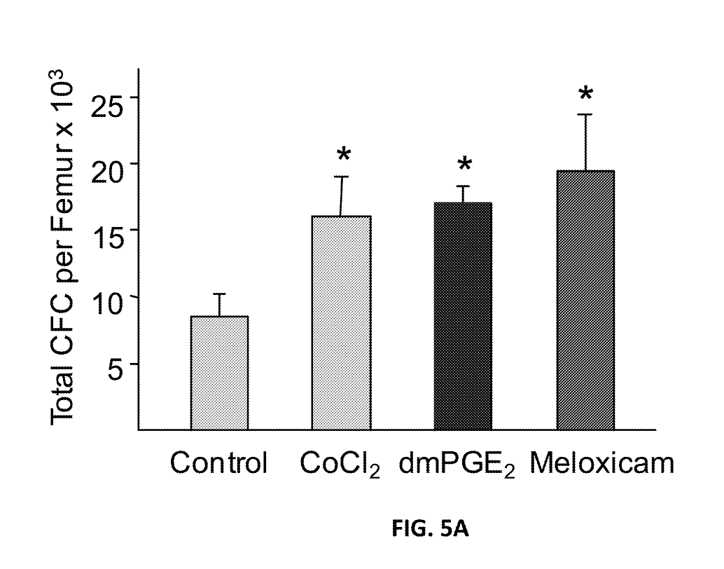

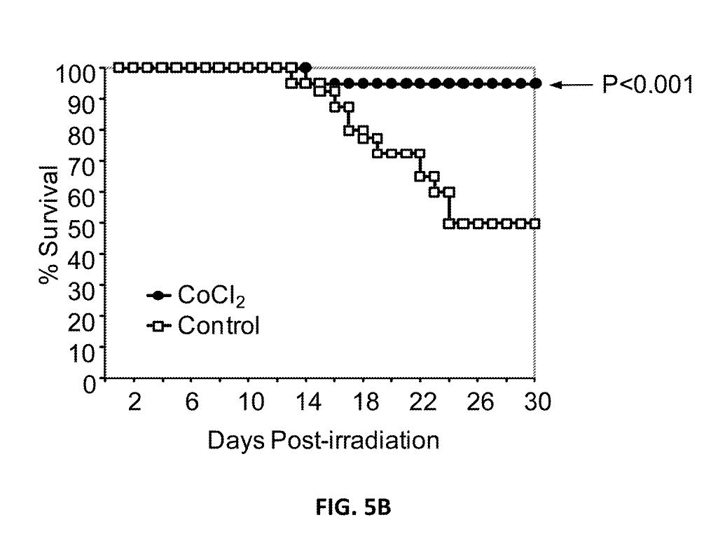

Briefly, irradiated mice were treated with a single subcutaneous dose of dmPGE₂ or vehicle control at 6 hours post-irradiation and moribund status and mortality were monitored for 30 days post-irradiation. Referring now to FIG. 2A, a single treatment with dmPGE₂ at 6 hours post-irradiation resulted in 95% survival (P=0.0011) compared to 50% survival in control mice. In normal, non-irradiated mice, total CFC(CFU-GM +BFU-E+CFU-GEMM) are generally in the range of 40,000 per femur. Referring now to FIG. 5A. a significant deficit in marrow HPC is evident in control mice that received 796 cGys and survived to day 35. However, in mice treated with dmPGE₂, marrow, HPC were still lower than historical controls but were significantly higher than control irradiated mice (FIG. 5A), indicating that hematopoiesis is more robust, likely as a result of enhanced stem cell repair, self-renewal and HSC and HPC expansion. These results indicate that dmPGE₂ is a highly effective radiomitigator.

Radiomitigation with Delayed NSAID Administration

As illustrated herein, exposure to PGE₂ early post-irradiation increases survival. However, exposure to PGE₂ is inhibitory to HPC expansion (Gentile and Pelus, 1988; Pelus and Gentile, 1988; Kurland et al., 1978; Kurland et al., 1979; Pelus et al., 1979; Pelus et al., 1981; Pelus et al., 1983; Pelus et al., 1988). And, as discussed earlier, the bodies of animals exposed to high level of ionizing radiation tend to increase the amount of PGE₂ that they produce. Dosing a patient with a compound that interferes with either the production of PGE₂ or with its effect on HPC expansion can obviate the negative effect of excess PGE₂ production. Reducing PGE₂ biosynthesis with a delayed administration of an NSAID, HPC inhibitory signaling by PGE₂ would be ablated and rapid hematopoietic expansion could occur, allowing for repopulation of the irradiated animal. Meloxicam administered to non-irradiated mice can increase HPCs within the bone marrow, leading to an increase in mature blood cells. To assess if meloxicam administration post-irradiation would lead to increased survival, irradiated mice were treated with 6 mg/kg meloxicam dosed once daily on days 2 through 5 post-irradiation and moribund status and mortality were monitored for 30. This delayed regimen of meloxicam post-irradiation resulted in 80% survival (P=0.034) compared to 50% survival in control mice (FIG. 5A). Similarly to the analysis of dmPGE₂ treatment, total CFC content in femurs 35 days post-irradiation was evaluated as a measure of hematopoietic recovery. Meloxicam administration significantly increased bone marrow CFC content compared to control, indicating that delayed NSAID administration post-irradiation can expand the hematopoietic compartment and increase survival in treated animals.

### Cobalt Chloride Administration Increases Survival Post-Irradiation

PGE₂ is a known transcriptional inducer of HIF-1α (Kaidi et al., 2006; Fukuda et al., 2003; Jung et al., 2003) and stabilizes HIF-1α protein (Piccoli et al., 2007; Liu et al., 2002). HIF-1α is a key transcriptional regulator with a broad repertoire of downstream target genes and is responsible for physiological adaptation from normoxia (21% O₂) to hypoxia (1% O₂) (reviewed in (Ke and Costa, 2006)). HIF-1α up-regulates EPO production (Semenza et al., 1991), the anti-apoptotic protein Survivin (Peng et al., 2006; Wei et al., 2006; Yang et al., 2004), numerous cell proliferation and survival genes (Feldser et al., 1999; Cormier-Regard et al., 1998; Krishnamachary et al., 2003), the angiogenic growth factor VEGF (Levy et al., 1995) and others. The HSC bone marrow niche is hypoxic (Levesque et al., 2007), and it has been suggested that this hypoxic niche maintains HIF-1α activity that maintains stem cells (Lin et al., 2006). Hypoxic conditions expand human HSC (Danet et al., 2003) and HPC (Smith and Broxmeyer, 1986; Broxmeyer et al., 1989; Broxmeyer et al., 1990) in vitro, creating a role for HIF-1α in HSC maintenance. In addition, HIF-1α has recently been reported to prevent hematopoietic cell damage caused by overproduction of reactive oxygen species (ROS) (Kirito et al., 2009). While the damaging effects of radiation exposure have largely been attributed to direct DNA damage (Hall, 2000a), it is now well recognized that the radiation damaging effects on HSC are also mediated by other stress response pathways, including oxidative stress. ROS have been implicated in mediating chronic oxidative stress resulting from radiation-induced late morbidity in long-term cancer survivors (Zhao et al., 2007). Oxidative stress-mediated radiation injury of hematopoietic (Nunia et al., 2007) and non-hematopoietic cells (Ishii et al., 2007; Wan et al., 2006) and the use of free radical scavengers to reverse the damage has been previously documented (Ishii et al., 2007; Wan et al., 2006; Sandhya et al., 2006; Rabbani et al., 2005). The reduction of ROS induced-damage, coupled with the up-regulation of erythropoiesis, angiogenesis, cell survival/proliferation, DNA repair and anti-apoptotic functions of HIF-1α, support a hypothesis of altering HIF-1α as a radiomitigation strategy.

Numerous radioprotectors, including dmPGE₂, cysteamine, 5-HT, and the FDA approved compound Amifostine, induce marrow hypoxia, and only doses of the compounds that induce sufficient hypoxia are radioprotective (Allalunis-Turner et al., 1989; Purdie et al., 1983; Glover et al., 1984). While the effects of Amifostine are traditionally thought to be due to free radical scavenging, it has been suggested that this induction of marrow hypoxia may play an important role for radioprotection (Kouvaris et al., 2007), further indicating that compounds which induce HIF-1α may be potent radioprotectors. Cobalt chloride (CoCl₂) is a known potent inducer and stabilizer of HIF-1α (Wang and Semenza, 1993; Yuan et al., 2003; Ke et al., 2005; Salnikow et al., 2004), and even before its mechanism of action was known, CoCl₂ was used to treat anemia in pregnant women, infants, and hemodialysis patients (Holly, 1955).

In one case, CoCl₂ was explored immediately after irradiation and was found to have positive effects on erythropoietic recovery and survival (Vittorio and Whitfield, 1971), yet its use as a radiomitigator is largely unexplored. As described herein, administering PGE₂ to an animal that has been exposed to high doses of ionizing radiation can have a positive effect on the animal's survival rates. This beneficial effect is especially pronounced if the compound is administered before the patient is treated with compounds that undo the initial beneficial effects of PGE₂ and similar molecules. Surprisingly, treating a patient with CoCl₂ has similar effects.

Briefly, irradiated mice were treated with a single subcutaneous dose of CoCl₂ (60 mg/kg) at 6 hours post-irradiation and moribund status and mortality were monitored as described. Single CoCl₂ treatment at 6 hours post-irradiation resulted in 95% survival (P=0.0011) (FIG. 5B.), similar to the effectiveness of dmPGE₂. In addition, as was the case with dmPGE₂ and meloxicam, increased survival in CoCl₂ treated mice correlated with significantly increased CFC in bone marrow (FIG. 5A.), indicating that CoCl₂ increased hematopoietic recovery and expansion post-irradiation.

### REFERENCES

Allalunis-Turner, M. J., Walden, T. L., Jr., and Sawich, C. (1989). Induction of marrow hypoxia by radioprotective agents. Radiat. Res. 118, 581-586.
Anno, G. H., Baum, S. J., Withers, H. R., and Young, R. W. (1989). Symptomatology of acute radiation effects in humans after exposure to doses of 0.5-30 Gy. Health Phys. 56, 821-838.
Broxmeyer, H. E., Cooper, S., and Gabig, T. (1989). The effects of oxidizing species derived from molecular oxygen on the proliferation in vitro of human granulocyte-macrophage progenitor cells. Ann. N.Y. Acad. Sci. 554, 177-184.
Broxmeyer, H. E., Cooper, S., Lu, L., Miller, M. E., Langefeld, C. D., and Ralph, P. (1990). Enhanced stimulation of human bone marrow macrophage colony formation in vitro by recombinant human macrophage colony-stimulating factor in agarose medium and at low oxygen tension. Blood 76, 323-329.
Broxmeyer, H. E., Galbraith, P. R., and Baker, F. L. (1976). Relationship of colony-stimulating activity to apparent kill of human colony-forming cells by irradiation and hydroxyurea. Blood 47, 403-411.
Broxmeyer, H. E., Mejia, J. A., Hangoc, G., Barese, C., Dinauer, M., and Cooper, S. (2007). SDF-1/CXCL12 enhances in vitro replating capacity of murine and human multipotential and macrophage progenitor cells. Stem Cells Dev. 16, 589-596.
Broxmeyer, H. E. and Smith, S. (2009). Cord blood stem cell transplantation. In Thomas' Hematopoietic Cell Transplantation, F. R. Appelbaum, S. J. Forman, R. S. Negrin, and K. G. Blume, eds. (Oxford: Wiley-Blackwell), pp. 559-576.
Cayeux, S. J., Beverley, P. C., Schulz, R., and Dorken, B. (1993). Elevated plasma prostaglandin E2 levels found in 14 patients undergoing autologous bone marrow or stem cell transplantation. Bone Marrow Transplant 12, 603-608.
Chen, X., Gresham, A., Morrison, A., and Pentland, A. P. (1996). Oxidative stress mediates synthesis of cytosolic phospholipase A2 after UVB injury. Biochim Biophys Acta 1299, 23-33.
Chinsoo, C. L. and Glatstein, E. (1998). Radiation Injury. In Harrison's Principles of Internal Medicine, A. S. Fauci, E. Braunwald, and K. L. Isselbacher, eds. (New York: McGraw-Hill), p. 2559.
Coleman, C. N., Blakely, W. F., Fike, J. R., MacVittie, T. J., Metting, N. F., Mitchell, J. B., Moulder, J. E., Preston, R. J., Seed, T. M., Stone, H. B., Tofilon, P. J., and Wong, R. S. (2003). Molecular and cellular biology of moderate-dose (1-10 Gy) radiation and potential mechanisms of radiation protection: report of a workshop at Bethesda, Md., Dec. 17-18, 2001. Radiat. Res. 159, 812-834.
Cormier-Regard, S., Nguyen, S. V., and Claycomb, W. C. (1998). Adrenomedullin gene expression is developmentally regulated and induced by hypoxia in rat ventricular cardiac myocytes. J. Biol. Chem. 273, 17787-17792.
Dainiak, N., Waselenko, J. K., Armitage, J. O., MacVittie, T. J., and Farese, A. M. (2003). The hematologist and radiation casualties. Hematology. Am. Soc. Hematol. Educ. Program. 473-496.
Danet, G. H., Pan, Y., Luongo, J. L., Bonnet, D. A., and Simon, M. C. (2003). Expansion of human SCID-repopulating cells under hypoxic conditions. J. Clin. Invest 112, 126-135.
Dominici, M., Rasini, V., Bussolari, R., Chen, X., Hofmann, T. J., Spano, C., Bernabei, D., Veronesi, E., Bertoni, F., Paolucci, P., Conte, P., and Horwitz, E. M. (2009). Restoration and reversible expansion of the osteoblastic hematopoietic stem cell niche after marrow radioablation. Blood 114, 2333-2343.
Feldser, D., Agani, F., Iyer, N. V., Pak, B., Ferreira, G., and Semenza, G. L. (1999). Reciprocal positive regulation of hypoxia-inducible factor 1alpha and insulin-like growth factor 2. Cancer Res. 59, 3915-3918.
Fukuda, R., Kelly, B., and Semenza, G. L. (2003). Vascular endothelial growth factor gene expression in colon cancer cells exposed to prostaglandin E2 is mediated by hypoxia-inducible factor 1. Cancer Res. 63, 2330-2334.
Fukuda, S., Bian, H., King, A. G., and Pelus, L. M. (2007). The chemokine GRObeta mobilizes early hematopoietic stem cells characterized by enhanced homing and engraftment. Blood 110, 860-869.
Gentile, P. S, and Pelus, L. M. (1988). In vivo modulation of myelopoiesis by prostaglandin E2. IV. Prostaglandin E2 induction of myelopoietic inhibitory activity. J. Immunol. 141, 2714-2720.
Glover, D., Negendank, W., Delivoria-Papadopoulos, M., and Glick, J. H. (1984). Alterations in oxygen transport following WR-2721. Int. J. Radiat. Oncol. Biol. Phys. 10, 1565-1568.
Hall, E. J. (2000a). Acute effects of total body iradiation. In Radiobiology for the Radiologist, E. J. Hall, ed. (Philadelphia: Lippincott, Williams & Wilkins), pp. 124-135.
Hall, E. J. (2000b). Radiobiology for the Radiologist. (Philadelphia: Lippincott).
Hanson, W. R. (1987). Radiation protection of murine intestine by WR-2721, 16,16-dimethyl prostaglandin E2, and the combination of both agents. Radiat. Res. 111, 361-373.
Hanson, W. R. and Ainsworth, E. J. (1985). 16,16-Dimethyl prostaglandin E2 induces radioprotection in murine intestinal and hematopoietic stem cells. Radiat. Res. 103, 196-203.
Herodin, F., Bourin, P., Mayol, J. F., Lataillade, J. J., and Drouet, M. (2003). Short-term injection of antiapoptotic cytokine combinations soon after lethal gamma-irradiation promotes survival. Blood 101, 2609-2616.
Herodin, F. and Drouet, M. (2005). Cytokine-based treatment of accidentally irradiated victims and new approaches. Exp. Hematol. 33, 1071-1080.
Hofer, M., Pospisil, M., Znojil, V., Hola, J., Vacek, A., Weiterova, L., Streitova, D., and Kozubik, A. (2006). Meloxicam, a cyclooxygenase 2 inhibitor, supports hematopoietic recovery in gamma-irradiated mice. Radiat. Res. 166, 556-560.
Holly, R. G. (1955). Studies on iron and cobalt metabolism. J. Am. Med. Assoc. 158, 1349-1352.
Ishii, J., Natsume, A., Wakabayashi, T., Takeuchi, H., Hasegawa, H., Kim, S. U., and Yoshida, J. (2007). The free-radical scavenger edaravone restores the differentiation of human neural precursor cells after radiation-induced oxidative stress. Neurosci. Lett. 423, 225-230.
Isoherranen, K., Punnonen, K., Jansen, C., and Uotila, P. (1999). Ultraviolet irradiation induces cyclooxygenase-2 expression in keratinocytes. Br J Dermatol 140, 1017-1022.
Jiao, W., Kiang, J. G., Cary, L., Elliott, T. B., Pellmar, T. C., and Ledney, G. D. (2009). COX-2 inhibitors are contraindicated for treatment of combined injury. Radiat. Res. 172, 686-697.
Jung, Y. J., Isaacs, J. S., Lee, S., Trepel, J., and Neckers, L. (2003). IL-1beta-mediated up-regulation of HIF-alpha via an NFkappaB/COX-2 pathway identifies HIF-1 as a critical link between inflammation and oncogenesis. FASEB J. 17, 2115-2117.
Kaidi, A., Qualtrough, D., Williams, A. C., and Paraskeva, C. (2006). Direct transcriptional up-regulation of cyclooxygenase-2 by hypoxia-inducible factor (HIF)-1 promotes colorectal tumor cell survival and enhances HIF-1 transcriptional activity during hypoxia. Cancer Res. 66, 6683-6691.
Kargacin, B. and Kostial, K. (1985). Reduction of 85Sr, 137Cs, 131I and 141Ce retention in rats by simultaneous oral administration of calcium alginate, ferrihexacyanoferrate(II), KI and Zn-DTPA. Health Phys. 49, 859-864.
Ke, Q. and Costa, M. (2006). Hypoxia-inducible factor-1 (HIF-1). Mol. Pharmacol. 70, 1469-1480.
Ke, Q., Kluz, T., and Costa, M. (2005). Down-regulation of the expression of the FIH-1 and ARD-1 genes at the transcriptional level by nickel and cobalt in the human lung adenocarcinoma A549 cell line. Int. J. Environ. Res. Public Health 2, 10-13.
Kirito, K., Hu, Y., and Komatsu, N. (2009). HIF-1 prevents the overproduction of mitochondrial ROS after cytokine stimulation through induction of PDK-1. Cell Cycle 8, 2844-2849.
Kouvaris, J. R., Kouloulias, V. E., and Vlahos, L. J. (2007). Amifostine: the first selective-target and broad-spectrum radioprotector. Oncologist. 12, 738-747.
Kozubik, A., Hofmanova, J., Pospisil, M., Netikova, J., Hola, J., and Lojek, A. (1994). Effects of drugs inhibiting prostaglandin or leukotriene biosynthesis on postirradiation haematopoiesis in mouse. Int. J. Radiat. Biol. 65, 369-377.
Krishnamachary, B., Berg-Dixon, S., Kelly, B., Agani, F., Feldser, D., Ferreira, G., Iyer, N., LaRusch, J., Pak, B., Taghavi, P., and Semenza, G. L. (2003). Regulation of colon carcinoma cell invasion by hypoxia-inducible factor 1. Cancer Res. 63, 1138-1143.
Kurland, J. I., Broxmeyer, H. E., Pelus, L. M., Bockman, R. S., and Moore, M. A. (1978). Role for monocyte-macrophage-derived colony-stimulating factor and prostaglandin E in the positive and negative feedback control of myeloid stem cell proliferation. Blood 52, 388-407.
Kurland, J. I., Pelus, L. M., Ralph, P., Bockman, R. S., and Moore, M. A. (1979). Induction of prostaglandin E synthesis in normal and neoplastic macrophages: role for colony-stimulating factor(s) distinct from effects on myeloid progenitor cell proliferation. Proc. Natl. Acad. Sci. U.S.A 76, 2326-2330.
Levesque, J. P., Winkler, I. G., Hendy, J., Williams, B., Helwani, F., Barbier, V., Nowlan, B., and Nilsson, S. K. (2007). Hematopoietic progenitor cell mobilization results in hypoxia with increased hypoxia-inducible transcription factor-1 alpha and vascular endothelial growth factor A in bone marrow. Stem Cells 25, 1954-1965.
Levy, A. P., Levy, N. S., Wegner, S., and Goldberg, M. A. (1995). Transcriptional regulation of the rat vascular endothelial growth factor gene by hypoxia. J. Biol. Chem. 270, 13333-13340.
Lin, Q., Lee, Y. J., and Yun, Z. (2006). Differentiation arrest by hypoxia. J. Biol. Chem. 281, 30678-30683.
Liu, X. H., Kirschenbaum, A., Lu, M., Yao, S., Dosoretz, A., Holland, J. F., and Levine, A. C. (2002). Prostaglandin E2 induces hypoxia-inducible factor-1alpha stabilization and nuclear localization in a human prostate cancer cell line. J Biol Chem 277, 50081-50086.
MacVittie, T. J., Farese, A. M., and Jackson, W., III (2005). Defining the full therapeutic potential of recombinant growth factors in the post radiation-accident environment: the effect of supportive care plus administration of G-CSF. Health Phys. 89, 546-555.
Moore, A. H., Olschowka, J. A., Williams, J. P., Okunieff, P., and O'Banion, M. K. (2005). Regulation of prostaglandin E2 synthesis after brain irradiation. Int J. Radiat. Oncol Biol Phys. 62, 267-272.
Moulder, J. E. (2004). Post-irradiation approaches to treatment of radiation injuries in the context of radiological terrorism and radiation accidents: a review. Int. J. Radiat. Biol. 80, 3-10.
Nishiguchi, I., Furuta, Y., Hunter, N., Murray, D., and Milas, L. (1990). Radioprotection of hematopoietic tissues in mice by indomethacin. Radiat. Res. 122, 188-192.
Nunia, V., Sancheti, G., and Goyal, P. K. (2007). Protection of Swiss albino mice against whole-body gamma irradiation by diltiazem. Br. J. Radiol. 80, 77-84.
Ogawa, M. (1993). Differentiation and proliferation of hematopoietic stem cells. Blood 81, 2844-2853.
Pellmar, T. C. and Rockwell, S. (2005). Priority list of research areas for radiological nuclear threat countermeasures. Radiat. Res. 163, 115-123.
Pelus, L. M., Broxmeyer, H. E., Kurland, J. I., and Moore, M. A. (1979). Regulation of macrophage and granulocyte proliferation. Specificities of prostaglandin E and lactoferrin. J. Exp. Med. 150, 277-292.
Pelus, L. M., Broxmeyer, H. E., and Moore, M. A. (1981). Regulation of human myelopoiesis by prostaglandin E and lactoferrin. Cell Tissue Kinet. 14, 515-526.
Pelus, L. M. and Gentile, P. S. (1988). In vivo modulation of myelopoiesis by prostaglandin E2. III. Induction of suppressor cells in marrow and spleen capable of mediating inhibition of CFU-GM proliferation. Blood 71, 1633-1640.
Pelus, L. M., Gold, E., Saletan, S., and Coleman, M. (1983). Restoration of responsiveness of chronic myeloid leukemia granulocyte-macrophage colony-forming cells to growth regulation in vitro following preincubation with prostaglandin E. Blood 62, 158-165.
Pelus, L. M., Ottmann, O. G., and Nocka, K. H. (1988). Synergistic inhibition of human marrow granulocyte-macrophage progenitor cells by prostaglandin E and recombinant interferon-alpha, -beta, and -gamma and an effect mediated by tumor necrosis factor. J. Immunol. 140, 479-484.
Peng, X. H., Karna, P., Cao, Z., Jiang, B. H., Zhou, M., and Yang, L. (2006). Cross-talk between epidermal growth factor receptor and hypoxia-inducible factor-1alpha signal pathways increases resistance to apoptosis by up-regulating survivin gene expression. J. Biol. Chem. 281, 25903-25914.
Piccoli, C., D'Aprile, A., Ripoli, M., Scrima, R., Boffoli, D., Tabilio, A., and Capitanio, N. (2007). The hypoxia-inducible factor is stabilized in circulating hematopoietic stem cells under normoxic conditions. FEBS Lett 581, 3111-3119.
Pospisil, M., Netikova, J., Kozubik, A., and Pipalova, I. (1989). Effect of indomethacin, diclofenac sodium and sodium salicylate on peripheral blood cell counts in sublethally gamma-irradiated mice. Strahlenther. Onkol. 165, 627-631.
Poston, J. W., Sr. (2005). Warren K. Sinclair Keynote Address: Current challenges in countering radiological terrorism. Health Phys. 89, 450-456.
Purdie, J. W., Inhaber, E. R., Schneider, H., and Labelle, J. L. (1983). Interaction of cultured mammalian cells with WR-2721 and its thiol, WR-1065: implications for mechanisms of radioprotection. Int. J. Radiat. Biol. Relat Stud. Phys. Chem. Med. 43, 517-527.
Rabbani, Z. N., Anscher, M. S., Folz, R. J., Archer, E., Huang, H., Chen, L., Golson, M. L., Samulski, T. S., Dewhirst, M. W., and Vujaskovic, Z. (2005). Overexpression of extracellular superoxide dismutase reduces acute radiation induced lung toxicity. BMC. Cancer 5, 59.
Salnikow, K., Donald, S. P., Bruick, R. K., Zhitkovich, A., Phang, J. M., and Kasprzak, K. S. (2004). Depletion of intracellular ascorbate by the carcinogenic metals nickel and cobalt results in the induction of hypoxic stress. J. Biol. Chem. 279, 40337-40344.
Sandhya, T., Lathika, K. M., Pandey, B. N., Bhilwade, H. N., Chaubey, R. C., Priyadarsini, K. I., and Mishra, K. P. (2006). Protection against radiation oxidative damage in mice by Triphala. Mutat. Res. 609, 17-25.
Semenza, G. L., Nejfelt, M. K., Chi, S. M., and Antonarakis, S. E. (1991). Hypoxia-inducible nuclear factors bind to an enhancer element located 3′ to the human erythropoietin gene. Proc. Natl. Acad. Sci. U.S.A 88, 5680-5684.
Serushago, B. A., Tanaka, K., Koga, Y., Taniguchi, K., and Nomoto, K. (1987). Positive effects of indomethacin on restoration of splenic nucleated cell populations in mice given sublethal irradiation. Immunopharmacology 14, 21-26.
Shaheen, M. and Broxmeyer, H. E. (2009). The humoral regulation of hematopoiesis. In Hematology: Basic Principles and Practice, R. Hoffman, E. J. J. Benz, S. J. Shattil, B. Furie, L. E. Silberstein, P. McGlave, H. Heslop, and J. Anastasi, eds. (Philadelphia: Elsevier Churchill Livingston), pp. 253-275.
Siegal, T. and Pfeffer, M. R. (1995). Radiation-induced changes in the profile of spinal cord serotonin, prostaglandin synthesis, and vascular permeability. Int J. Radiat. Oncol Biol Phys. 31, 57-64.
Smith, S. and Broxmeyer, H. E. (1986). The influence of oxygen tension on the long-term growth in vitro of haematopoietic progenitor cells from human cord blood. Br. J. Haematol. 63, 29-34.
Till, J. E. and McCulloch, E. A. (1964). Repair Processes in Irradiated Mouse Hematopoietic Tissue. Ann. N.Y. Acad. Sci. 114, 115-125.
Vittorio, P. V. and Whitfield, J. F. (1971). The beneficial effects of postirradiation treatment with cobaltous chloride on the development of nuclear damage in thymocytes, erythropoiesis, and the survival of mice. Can. J. Physiol Pharmacol. 49, 812-818.
Wald, N. (1982). Radiation Injury. In Cecil Textbook of Medicine, J. B. Wyngaarden and L. H. J. Smith, eds. (Philadelphia: W.B. Saunders), p. 2228.
Walden, T. L., Jr. and Farzaneh, N. K. (1995). Radioprotection by 16,16 dimethyl prostaglandin E2 is equally effective in male and female mice. J. Radiat. Res. (Tokyo) 36, 1-7.
Walden, T. L., Jr., Patchen, M., and Snyder, S. L. (1987). 16,16-Dimethyl prostaglandin E2 increases survival in mice following irradiation. Radiat. Res. 109, 440-448.
Wan, X. S., Ware, J. H., Zhou, Z., Donahue, J. J., Guan, J., and Kennedy, A. R. (2006). Protection against radiation-induced oxidative stress in cultured human epithelial cells by treatment with antioxidant agents. Int. J. Radiat. Oncol. Biol. Phys. 64, 1475-1481.
Wang, G. L. and Semenza, G. L. (1993). General involvement of hypoxia-inducible factor 1 in transcriptional response to hypoxia. Proc. Natl. Acad. Sci. U. S. A 90, 4304-4308.
Wei, H., Wang, C., and Chen, L. (2006). Proliferating cell nuclear antigen, survivin, and CD34 expressions in pancreatic cancer and their correlation with hypoxia-inducible factor 1alpha. Pancreas 32, 159-163.
Wen, Q., Leung, C., Huang, Z., Small, S., Reddi, A. L., Licht, J. D., and Crispino, J. D. (2009). Survivin is not required for the endomitotic cell cycle of megakaryocytes. Blood 114, 153-156.
Yang, L., Cao, Z., Li, F., Post, D. E., Van Meir, E. G., Zhong, H., and Wood, W. C. (2004). Tumor-specific gene expression using the survivin promoter is further increased by hypoxia. Gene Ther. 11, 1215-1223.
Yuan, Y., Hilliard, G., Ferguson, T., and Millhorn, D. E. (2003). Cobalt inhibits the interaction between hypoxia-inducible factor-alpha and von Hippel-Lindau protein by direct binding to hypoxia-inducible factor-alpha. J. Biol. Chem. 278, 15911-15916.
Zhao, W., Diz, D. I., and Robbins, M. E. (2007). Oxidative damage pathways in relation to normal tissue injury. Br. J. Radiol. 80 Spec No 1, S23-S31.

While the novel technology has been illustrated and described in detail in the figures and foregoing description, the same is to be considered as illustrative and not restrictive in character; it being understood that only the preferred embodiments have been shown and described and that all changes and modifications that come within the spirit of the novel technology are desired to be protected. As well, while the novel technology was illustrated using specific examples, theoretical arguments, accounts, and illustrations, these illustrations and the accompanying discussion should by no means be interpreted as limiting the technology. All patents, patent applications, and references to texts, scientific treatises, publications, and the like referenced in this application are incorporated herein by reference in their entirety.

1. A method of treating radiation poisoning, comprising the steps of:
administering a therapeutically effective dose of PGE₂ or of a compound that has the same or a similar physiological effect as PGE₂ to a patient soon after said patient has been exposed to a pathological dose of ionizing radiation; and
treating the patient with a therapeutically effective course of treatment that reduces the activity of PGE₂, wherein the course of treatment includes at least one compound that reduces the activity of PGE₂, and wherein there is a delay between the step of administering the therapeutically effective dose of PGE₂ and the step of treating the patient the course of treatment that reduces the activity of PGE₂.

2. The method according to claim 1, wherein the therapeutically effective course of treatment that reduces PGE₂ activity lasts for at least 4 days.

3. The method according to claim 1, wherein said effective dose of PGE₂ or a compound that has the same effect as PGE₂ is administered within 24 hours of the patient's exposure to the dose of ionizing radiation.

4. The method according to claim 1, wherein said effective dose of PGE₂ or a compound that has the same effect as PGE₂ is administered within 12 hours of the patient's exposure to the dose of ionizing radiation.

5. The method according to claim 1, wherein said effective dose of PGE₂ or a compound that has the same effect as PGE₂ is administered within about 6 hours of the patient's exposure to the dose of ionizing radiation.

6. The method according to claim 1, wherein the dose of at least one compound that reduces the activity of PGE₂ is given to the patient at least about 24 hours after the patient has been exposed to the pathological dose of ionizing radiation.

7. The method according to claim 1, wherein the dose of at least one compound that reduces the production of or efficacy of PGE₂ is given to the patient at least about 36 hours after the patient has been exposed to the pathological dose of ionizing radiation.

8. The method according to claim 1, wherein the treatment course that includes the at least one compound that reduces the production of or efficacy of PGE₂ is given to the patient at least about 48 hours after the patient has been exposed to the pathological dose of ionizing radiation.

9. The method according to claim 1, wherein the compound that has the same or similar activity as PGE₂ is dmPGE₂ or a pharmaceutically acceptable salt thereof.

10. The method according to claim 1, wherein the therapeutically effective dose of PGE₂ or a compound that has the same or similar activity to PGE₂ includes at least one compound selected from the group consisting of: PGE₁, PGE₃, dmPGE₁, and dmPGE₃.

11. The method according to claim 9, wherein said therapeutically effective dose of dmPGE₂ is in the range of about 10.0 to about 0.01 mg per kg⁻¹.

12. The method according to claim 9, wherein said therapeutically effective does of dmPGE₂ is in the range of about 5.0 to about 0.08 mg per kg⁻¹.

13. The method according to claim 9, wherein said therapeutically effective does of dmPGE₂ is in the range of about 2.0 to about 0.1 mg per kg⁻¹.

14. The method according to claim 1, wherein the at least one compound that reduces the activity of PGE₂ is a non-steroidal anti-inflammatory compound.

15. The method according to claim 14, wherein the non-steroidal anti-inflammatory acts on at least one enzyme selected from the group consisting of: cyclooxygenase-1 and cyclooxygenase-2.

16. The method according to claim 14, wherein the non-steroidal anti-inflammatory compound acts primarily on cyclooxygenase-2.

17. The method according to claim 14, wherein the non-steroidal anti-inflammatory compound is selected from the group consisting of: aspirin, celecoxib, rofecoxib, etoricoxib, valdecoxib, ibuprofen, naproxen, diclofenac, etodolac, ketorolac, indomethacin, meloxicam and licofelone.

18. The method according to claim 14, wherein the non-steroidal anti-inflammatory compound is indomethacin or a pharmaceutically acceptable salt thereof.

19. The method according to claim 14, wherein the therapeutically effective dose of said non-steroidal anti-inflammatory compound is selected from one of the following dosages about 100 to about 0.01 mg per kg⁻¹ per day and about 75 to about 0.05 mg per kg⁻¹ per day.

20. The method according to claim 14, wherein the therapeutically effective dose of said non-steroidal anti-inflammatory compound is about 75 to about 0.05 mg per kg⁻¹ per day.

21. The method according to claim 14, wherein the non-steroidal anti-inflammatory compound is meloxicam or a pharmaceutically acceptable salt thereof.

22. The method according to claim 21, wherein the therapeutically effective dose of meloxicam is about to about 75 to about 0.05 mg per kg⁻¹ per day.

23. The method according to claim 21, wherein the therapeutically effective dose of meloxicam is about to about 50 to about 1.0 mg per kg⁻¹ per day.

24. The method according to claim 21, wherein the therapeutically effective dose of meloxicam is about to about 6.0 to about 0.1 mg per kg⁻¹ per day.

25. The method according to claim 1, wherein the compound that alters the activity of PGE₂ is an antagonist of at least one PGE₂ receptor.

26. The method according to claim 25, wherein the antagonist of at least one PGE₂ receptor is selected from the groups consisting of: N-[[4′-[[3-butyl-1,5-dihydro-5-oxo-1-[2-(trifluoromethyl)phenyl]-4H-1,2,4-triazol-4-yl]methyl][1,1′-biphenyl]-2-yl]sulfonyl]-3-methyl-2-thiophenecarboxamide and 4-(4,9-diethoxy-1,3-dihydro-1-oxo-2H-benz[f]isoindol-2-yl)-N-(phenylsulfonyl)-benzeneacetamide.

27. A method of treating radiation poisoning, comprising the steps of:
administering a therapeutically effective course of treatment, the course including doses of at least one compound that increases Hif-1α activity.

28. The method according to claim 27, wherein said course of treatment with the compound that increases Hif-1α activity is started within 6 hours of the patient's exposure to ionizing radiation.

29. The method according to claim 27, wherein said course of treatment with the compound that increases Hif-1α activity is started within 12 hours of the patient's exposure to ionizing radiation.

30. The method according to claim 27, wherein said course of treatment with the compound that increases Hif-1α activity is started within 24 hours of the patient's exposure to ionizing radiation.

31. The method according to claim 27, wherein said course of treatment with the compound that increases Hif-1α activity is started at least after 24 hours of the patient's exposure to ionizing radiation.

32. The method according to claim 27, wherein the course of treatment lasts for at least about 2 days.

33. The method according to claim 27, wherein the course of treatment lasts from about 2 to about 7 days.

34. The method according to claim 27, wherein the compound that increases Hif-1α activity is CoCl₂.

35. The method according to claim 28, wherein the course of treatment is a single bolus dose.

36. The method according to claim 29, wherein the course of treatment is a single bolus dose.

37. The method according to claim 30, wherein the course of treatment is a single bolus dose.

38. The method according to claim 34, wherein the therapeutically effective dose of CoCl₂ is about between about 120.0 to about 5.0 mg per kg⁻¹ per day.

39. The method according to claim 34, wherein the therapeutically effective dose of CoCl₂ is about between about 100.0 to about 10.0 mg per kg⁻¹ per day.

40. The method according to claim 34, wherein the therapeutically effective dose of CoCl₂ is about between about 60.0 to about 20.0 mg per kg⁻¹ per day.
